(19) United States
(12) Patent Application Publication
(10) Pub. No.: US 2024/0181108 A1
(43) Pub. Date: Jun. 6, 2024

(54) Antiviral Resilient Flooring

(71) Applicant: M.J. INTERNATIONAL FLOORING AND INTERIOR PRODUCTS INC., New Taipei City (TW)

(72) Inventor: Ben-Yuan Chen, New Taipei City (TW)

(21) Appl. No.: 18/170,634

(22) Filed: Feb. 17, 2023

(30) Foreign Application Priority Data
Dec. 6, 2022 (TW) 111146811

(51) Int. Cl. A61L 2/232; E04F 15/18

(52) U.S. Cl. CPC A61L 2/232; E04F 15/18; E04F 2290/04

(57) ABSTRACT

An antiviral resilient flooring including a stone plastic core layer, a print layer laid on the core layer, a transparent wear layer laid on the print layer, and a scratch resistant surface coating layer on top. The transparent wear layer and the surface coating layer both contain 0.5 wt % to 5 wt % of an inorganic antiviral material.

### FIELD OF INVENTION

The present invention relates to a flooring, especially an antiviral resilient flooring.

### BACKGROUND OF THE INVENTION

Modern people spend a lot of time staying in indoor environments with limited spaces. According to research, a variety of viruses can survive on the surface of flooring for few days or even longer, so the flooring might be a hotbed for viruses and other pathogens. When viruses are attached to the surface of flooring, people who walk on the flooring might transfer the viruses to other places by stepping on the flooring with their feet or shoes, and babies might also be exposed to the viruses when lying or crawling on the flooring. In addition, air flow caused by air-conditioner or by moving objects might also raise and transport particles with viruses on the surface of a flooring, so the time and distance of the transmission of the viruses are increased.

To prevent viruses and other pathogens from attaching to the surface of a flooring and spreading diseases, the surface of a flooring needs to be cleaned and sterilized periodically, the flooring is mopped with disinfectants or spraying disinfectants on the flooring. However, the cleaning process takes a lot of time and effort, and it cannot continuously prevent the adhesion and transfer of viruses. Namely, viruses and other pathogens can still adhere and transfer between the intervals of the cleaning processes. Although some flooring contains an antibacterial coating or an antifungal coating, the floorings are merely antibacterial or antifungal with no antiviral functionality. Moreover, the antibacterial or antifungal function of the flooring is only effective for a short-term at the beginning. The flooring would gradually lose the antibacterial or antifungal ability due to the wear of the coating by external forces such as friction.

Therefore, it is an object of the present invention to provide a flooring with long-term antiviral functionality.

### SUMMARY OF THE INVENTION

A flooring with long-term antiviral functionality is achieved by an antiviral resilient flooring having:
a stone plastic core layer, wherein the stone plastic core layer comprises thermoplastics, fillers, plasticizers/process oils, stabilizers, and processing aids;
a print layer, wherein the print layer is on a top of the stone plastic core layer, and the print layer comprises an opaque thermoplastic layer with printed design or pattern; and
a transparent wear layer, wherein the transparent wear layer is on a top of the print layer, and the transparent wear layer comprises thermoplastics, plasticizers, stabilizers, and an inorganic antiviral material (antiviral glass powder); wherein
the antiviral resilient flooring is formed by the stone plastic core layer, the print layer, and the transparent wear layer. The layers are stacked in sequence and fused together by heat and pressure.

Wherein, the transparent wear layer comprises 0.5 wt % to 5 wt % of the inorganic antiviral material.

Wherein, a surface coating layer is coated and cured on a top of the transparent wear layer, wherein the surface coating layer comprises 0.5 wt % to 5 wt % of the inorganic antiviral material.

Wherein, the inorganic antiviral material comprises glass powders with 1 wt % to 2 wt % of silver nanoparticles.

Wherein, the glass powder comprises phosphate.

Wherein, the thickness of the transparent wear layer ranges from 150 micrometers to 700 micrometers.

Wherein, the thermoplastics can be polyvinyl chloride (PVC), polypropylene (PP), thermoplastic polyolefin (TPO), thermoplastic polyurethane (TPU), polyester (PET/PETG/PBT), or polycarbonate (PC).

Wherein, the thickness of the surface coating layer ranges from 5 micrometers to 20 micrometers.

Wherein, the surface coating layer comprises polyacrylate formed by ultraviolet light curing of acrylate monomers.

Many of the attendant features and advantages of the present invention will become better understood with reference to the following detailed description considered in connection with the accompanying figures and drawings.

### BRIEF DESCRIPTION OF THE DRAWINGS

The foregoing and other objects, aspects and advantages will be better understood from the following detailed description of a preferred embodiment of the invention with reference to the drawings, in which:

FIG. 1 is a perspective view of a first embodiment of an antiviral resilient flooring in accordance with the present invention,

FIG. 2 is a perspective view of a second embodiment of an antiviral resilient flooring in accordance with the present invention.

### DETAILED DESCRIPTION OF THE PREFERRED EMBODIMENTS

Reference will now be made in detail to the present preferred embodiments of the invention, examples of which are illustrated in the accompanying drawings. Wherever possible, the same reference numbers are used in the drawings and the description to refer to the same or like parts. It is not intended to limit the method by the exemplary embodiments described herein. In the following detailed description, for purposes of explanation, numerous specific details are set forth in order to attain a thorough understanding of the disclosed embodiments. It will be apparent, however, that one or more embodiments may be practiced without these specific details. As used in the description herein and throughout the claims that follow, the meaning of “a”, “an”, and “the” may include reference to the plural unless the context clearly dictates otherwise. Also, as used in the description herein and throughout the claims that follow, the terms “comprise or comprising”, “include or including”, “have or having”, “contain or containing” and the like are to be understood to be open-ended, i.e., to mean including but not limited to.

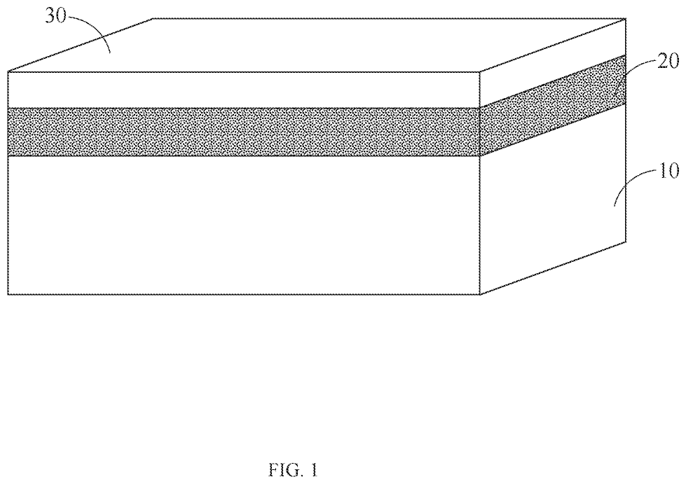

With reference to FIG. 1, a first embodiment of antiviral resilient flooring in accordance with the present invention from bottom to top comprises a stone plastic core layer 10, a print layer 20, and a transparent wear layer 30. The stone plastic core layer 10 is made of materials comprising thermoplastics, fillers, plasticizers/process oils, stabilizers, and processing additives. The manufacturing process of the stone plastic core layer 10 can be direct extrusion of said materials or first mixing and then calendaring of the said materials. The print layer 20 is formed on a top of the stone plastic core layer 10, and the print layer 20 is an opaque thermoplastic layer with printing design or pattern. The transparent wear layer 30 is formed on a top of the print layer 20, and the thickness of the transparent wear layer 30 is ranged from 150 micrometers to 700 micrometers. The transparent wear layer 30 comprises thermoplastics, plasticizers, stabilizers, and an inorganic antiviral material, wherein the transparent wear layer 30 contains 0.5 wt % to 5 wt % of the inorganic antiviral material. In a preferred embodiment, the thermoplastics for the transparent wear layer 30 includes polyvinyl chloride (PVC), and the plasticizer includes dioctyl terephthalate (DOTP), and the stabilizer includes calcium-zinc stabilizer and epoxidized soybean oil (ESBO). The transparent wear layer 30 contains 1 wt % of the inorganic antiviral material. The raw materials of the transparent wear layer 30 are mixed, calendared, and cooled to form the transparent wear layer 30. The stone plastic core layer 10, the print layer 20, and the transparent wear layer 30 are stacked in sequence and fused together to form a laminated plank with pressure and heat applied.

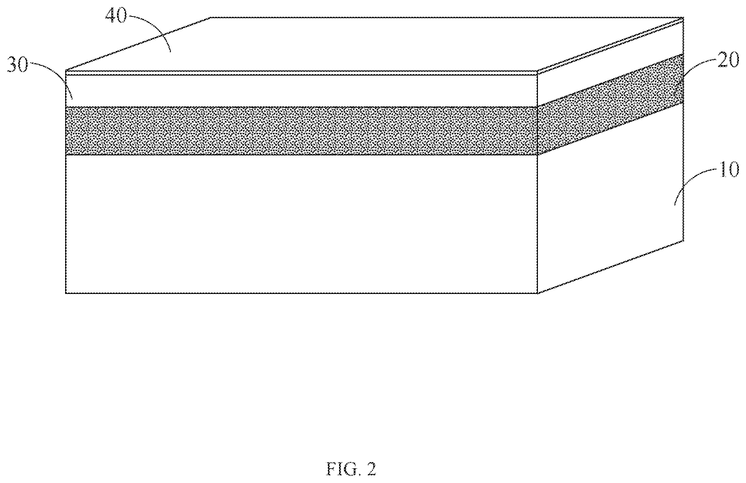

With reference to FIG. 2, in a second embodiment, the antiviral resilient flooring in accordance with the present invention from bottom to top comprises a stone plastic core layer 10, a print layer 20, a transparent wear layer 30, and a surface coating layer 40. The configurations of the stone plastic core layer 10, the print layer 20, and the transparent wear layer 30 are similar to those of the first embodiment, and the surface coating layer 40 is coated on the transparent wear layer 30. The surface coating layer 40 contains the inorganic antiviral material and polyacrylate formed by ultraviolet light curing of acrylate monomers. The thickness of the surface coating layer 40 is ranged from 5 micrometers to 20 micrometers. The surface coating layer 40 of the antiviral resilient flooring provides short-term enhancement of wear resistance and setup the surface gloss of the antiviral resilient flooring. Additional additives can be added to the surface coating layer 40 to enhance other functions of the antiviral resilient flooring. The transparent wear layer 30 contains 0.5 wt % to 5 wt % of the inorganic antiviral material, and the surface coating layer 40 also contains 0.5 wt % to 5 wt % of the inorganic antiviral material. In a preferred embodiment, the transparent wear layer 30 contains 1 wt % of the inorganic antiviral material, and the surface coating layer 40 also contains 1 wt % of the inorganic antiviral material. The order of structure for the stone plastic core layer 10, the print layer 20, and the transparent wear layer 30 of the second embodiment are similar to order of structure for the stone plastic core layer 10, the print layer 20, and the transparent wear layer 30 of the first embodiment. The surface coating layer 40 might further comprises 5 wt % to 20 wt % of silica as a matting agent. In one preferred embodiment, the inorganic antiviral material, and the liquid acrylate monomers with silica powder are mixed stirred together evenly to form a homogeneous liquid mixture, the mixture is coated on the transparent wear layer 30 and then cured with ultraviolet light to form the hard surface coating layer 40. The mixture is continuously circulated during the process to make sure the inorganic antiviral material and the silica power are evenly distributed in the mixture.

Said inorganic antiviral material of this invention comprises glass powders with 1 wt % to 2 wt % of silver nanoparticles. An average particle diameter (D50) of the inorganic antiviral material added in the transparent wear layer 30 is around 10 micrometers and D98 particle size of the glass powders of the inorganic antiviral material added in the transparent wear layer 30 is less than 40 micrometers. The D50 of the glass powders of the inorganic antiviral material added in the surface coating layer 40 is around 2 micrometers and D98 particle size of the inorganic antiviral material added in the surface coating layer 40 is less than 5 micrometers. In one embodiment, the inorganic antiviral material of this invention comprises phosphate with 1 wt % to 2 wt % of silver nanoparticles. Phosphate are slightly soluble in water, the phosphate in the glass powder near the surface of the antiviral resilient flooring can absorb some moisture in the atmosphere and the silver ions can be slightly dissolved and slowly released to the surface of the antiviral resilient flooring continuously to provide adequate dosage of the silver ions. The silver ions carry positive charges, and the cell walls of many pathogens possess negative charges, so the silver ions would attach to and break down the cell walls of the pathogens, the silver ions further penetrate into the cells and destroy the inner structures (mitochondria, vacuoles, and ribosomes) and biomolecules (proteins, lipids, and DNAs). The silver ions can disrupt the spike protein on the capsids of viruses and can interact with viral nucleic acids and thus antiviral. Said inorganic antiviral material can be mixed with other materials and distribute evenly in the transparent wear layer 30 and the surface coating layer 40 during the manufacturing process.

The antiviral resilient flooring of this invention has effective antiviral functionality, and the antiviral resilient flooring of this invention has greater antipathogenic properties than common antibacterial flooring or common mold resistant flooring. The antiviral resilient flooring of this invention also complies with flooring standards such as ASTM F1700, ASTM F3261, ISO 10582, ISO 20326, ISO 19322, JIS A 5705, GB/T 4085, GB/T 34440, and CNS 8906.

TABLE 1 shows the antiviral efficacies of the antiviral resilient flooring of the first embodiment with 1%, 2%, and 3% of said inorganic antiviral material added to the transparent wear layer 30. The antiviral resilient flooring of the first embodiment shows 99.99% of antiviral efficacy against influenza virus (H3N2). Based on same antiviral mechanism, the antiviral resilient flooring of this invention should show similar antiviral efficacy to other types of viruses such as the SARS-COV-2 virus.

TABLE 1

| Percentage of inorganic antiviral | Percentage of |
|---|---|
| material used in wear layer | antiviral efficacy |
| 3 wt % | 99.99% |
| 2 wt % | 99.99% |
| 1 wt % | 99.99% |

TABLE 2 shows the antibacterial efficacies according to ISO 22196-2011 specifications of the antiviral resilient flooring of the first embodiment with 1% of said inorganic antiviral material added to the transparent wear layer 30. The antiviral resilient flooring of the first embodiment shows 99.99% of antibacterial efficacies against Escherichia coli, Klebsiella pneumoniae, Methicillin-resistant Staphylococcus aureus (MRSA), and Pseudomonas aeruginosa, showing excellent antibacterial function.

TABLE 2

| | Percentage of |
|---|---|
| Test bacteria | antibacterial efficacy |
| Escherichia coli | 99.99% |
| Klebsiella pneumoniae | 99.99% |
| Methicillin-resistant Staphylococcus aureus | 99.99% |
| Pseudomonas aeruginosa | 99.99% |
| Staphylococcus aureus | 99.97% |

TABLE 3 shows the antifungal rating according to ASTM G21-15 specifications of the antiviral resilient flooring of the first embodiment with 1% of said inorganic antiviral material added to the transparent wear layer 30. The ratings of the antiviral resilient flooring of the first embodiment are 0, which means the antiviral resilient flooring of the first embodiment remains free of fungal growth after 28 days of incubation, an excellent antifungal function is shown. (A rating score from 0 to 4 is given based on the amount of growth that exists. The description of the rating system is as follows: 0=Specimen remained free of fungal growth; 1=Traces of growth on the specimen (less than 10%); 2=Light fungal growth on the specimen (10 to 30%); 3=Medium fungal growth on the specimen (30 to 60%); 4=Heavy fungal growth on the specimen (60% to complete coverage).)

TABLE 3

| Test fungi | Rating |
|---|---|
| Aspergillus brasiliensis | 0 |
| Aureobasidium pullulans | 0 |
| Chaetomium globosum | 0 |
| Trichoderma virens | 0 |
| Penicillium funiculosum | 0 |

According to TABLE 1, TABLE 2, and TABLE 3, the antiviral resilient flooring with the inorganic antiviral material added to the transparent wear layer 30 shows antiviral, antibacterial, and antifungal effects. With the inorganic antiviral material added, the surface coating layer 40 for short-term enhanced wear resistance would also possess similar antiviral, antibacterial, and antifungal effects. As the transparent wear layer 30 and the surface coating layer 40 contain the inorganic antiviral material, the antiviral resilient flooring of this invention has excellent long term antiviral and antipathogenic properties. Even if the antiviral resilient flooring of this invention has some wears under normal usages, the antiviral effect of the antiviral resilient flooring remains active due to the evenly distributed powders of the inorganic antiviral material within the transparent wear layer 30 and the surface coating layer 40 and thus ensures the presence of active ingredients on the surface of the antiviral resilient flooring. Even at a low concentration, the silver nanoparticles in the inorganic antiviral material have antiviral and antipathogenic effects. The antiviral and antipathogenic effects of the inorganic antiviral material do not decrease with time as those of organic antibacterial material, so long term and stable antiviral effect of the antiviral resilient flooring of this invention is ensured.

As above descriptions, the present invention has beneficial effects and advantages as follows:
1. Low concentration silver nanoparticles are used as antiviral agents, which are safer, more stable, and more enduring than organic antibacterial agents. The inorganic antiviral material used in this invention is FDA registered and EPA registered, and complies with The Biocidal Products Regulation No. 528/2012 of the European Union, ensuring a high level of safety for both human and animal health and the environment.
2. The thickness of the transparent wear layer 30 of the antiviral resilient flooring of this invention is 150 micrometers or thicker, with evenly distributed powders of the inorganic antiviral material in the transparent wear layer 30, high dosage of silver ions continuously presents on the surface of the antiviral resilient flooring after the inorganic antiviral material slowly dissolves when in contact with the moisture in the atmosphere, the antiviral resilient flooring of this invention possesses stable and long term antiviral and antipathogenic effects, the cost of time and labor to periodically sterilize and maintain the antiviral resilient flooring is minimized.
3. The transparent wear layer 30 of the antiviral resilient flooring of this invention is transparent. The transparent wear layer 30 includes a low but adequate concentration of transparent inorganic antiviral material powders, so the print layer 20 under the transparent wear layer 30 is still visible and the printing design or pattern of the antiviral resilient flooring of this invention is highly customizable without affecting the antiviral function.

The above specification, examples, and data provide a complete description of the present disclosure and use of exemplary embodiments. Although various embodiments of the present disclosure have been described above with a certain degree of particularity, or with reference to one or more individual embodiments, those with ordinary skill in the art could make numerous alterations or modifications to the disclosed embodiments without departing from the spirit or scope of this disclosure.

What is claimed is:

1. An antiviral resilient flooring comprising:
a stone plastic core layer, wherein the stone plastic core layer comprises thermoplastics, fillers, plasticizers/process oils, stabilizers, and processing additives;
a print layer is put on a top of the stone plastic core layer, wherein the print layer comprises an opaque thermoplastic layer with ink printed design or pattern; and
a transparent wear layer is formed on a top of the print layer, wherein the transparent wear layer comprises thermoplastics, plasticizers, stabilizers, and an inorganic antiviral material,
wherein the antiviral resilient flooring is formed by the stone plastic core layer, the print layer, and the transparent wear layer stacked in sequence and fused together with heat.

2. The antiviral resilient flooring of claim 1, wherein the transparent wear layer comprises 0.5 wt % to 5 wt % of the inorganic antiviral material.

3. The antiviral resilient flooring of claim 1, wherein a surface coating layer is coated and cured on a top of the transparent wear layer, wherein the surface coating layer comprises 0.5 wt % to 5 wt % of the inorganic antiviral material.

4. The antiviral resilient flooring of claim 1, wherein the inorganic antiviral material comprises glass powders with 1 wt % to 2 wt % of silver nanoparticles.

5. The antiviral resilient flooring of claim 2, wherein the inorganic antiviral material comprises glass powders with 1 wt % to 2 wt % of silver nanoparticles.

6. The antiviral resilient flooring of claim 3, wherein the inorganic antiviral material comprises glass powders with 1 wt % to 2 wt % of silver nanoparticles.

7. The antiviral resilient flooring of claim 4, wherein the glass powders comprise phosphates.

8. The antiviral resilient flooring of claim 5, wherein the glass powders comprise phosphates.

9. The antiviral resilient flooring of claim 6, wherein the glass powders comprise phosphates.

10. The antiviral resilient flooring of claim 1, wherein the thickness of the transparent wear layer ranges from 150 micrometers to 700 micrometers.

11. The antiviral resilient flooring of claim 2, wherein the thickness of the transparent wear layer ranges from 150 micrometers to 700 micrometers.

12. The antiviral resilient flooring of claim 3, wherein the thickness of the transparent wear layer ranges from 150 micrometers to 700 micrometers.

13. The antiviral resilient flooring of claim 4, wherein the thickness of the transparent wear layer ranges from 150 micrometers to 700 micrometers.

14. The antiviral resilient flooring of claim 5, wherein the thickness of the transparent wear layer ranges from 150 micrometers to 700 micrometers.

15. The antiviral resilient flooring of claim 5, wherein the thermoplastics includes but not limited to polyvinyl chloride (PVC), polypropylene (PP), thermoplastic polyolefin (TPO), thermoplastic polyurethane (TPU), polyester (PET/PETG/PBT), and polycarbonate (PC).

16. The antiviral resilient flooring of claim 3, wherein the thickness of the surface coating layer ranges from 5 micrometers to 20 micrometers.

17. The antiviral resilient flooring of claim 6, wherein the thickness of the surface coating layer ranges from 5 micrometers to 20 micrometers.

18. The antiviral resilient flooring of claim 3, wherein the surface coating layer comprises polyacrylates formed by ultraviolet light curing of acrylate monomers.

19. The antiviral resilient flooring of claim 16, wherein the surface coating layer comprises polyacrylates formed by ultraviolet light curing of acrylate monomers.

20. The antiviral resilient flooring of claim 17, wherein the surface coating layer comprises polyacrylates formed by ultraviolet light curing of acrylate monomers.
